(19) United States
(12) Patent Application Publication
(10) Pub. No.: US 2025/0320313 A1
(43) Pub. Date: Oct. 16, 2025

(54) METHOD FOR PREPARING POLY(IONIC LIQUID) (PIL)-FUNCTIONALIZED CELLULOSE CONDUCTIVE HYDROGEL

(71) Applicant: Shaanxi University of Science & Technology, Xi'an City (CN)

(72) Inventors: Sufeng ZHANG, Xi'an City (CN); Xue YAO, Xi'an City (CN); Ning WEI, Xi'an City (CN); Liwei QIAN, Xi'an City (CN); Hao DING, Xi'an City (CN); Jinrui LI, Xi'an City (CN)

(21) Appl. No.: 18/948,634

(22) Filed: Nov. 15, 2024

(30) Foreign Application Priority Data
Apr. 11, 2024 (CN) 202410435183.3

(51) Int. Cl. C08B 1/00

(52) U.S. Cl. CPC C08B 1/003

(57) ABSTRACT

Provided is a method for preparing a poly(ionic liquid) (PIL)-functionalized cellulose conductive hydrogel, including: step 1, preparing an ionic liquid monomer A and an ionic liquid monomer P; step 2, preparing a PIL: P(Aₓ-co-P_{y}) from the ionic liquid monomer A and the ionic liquid monomer obtained in step 1; and step 3, preparing the PIL-functionalized cellulose conductive hydrogel form the PIL obtained in step 2.

### CROSS REFERENCE TO RELATED APPLICATION

This patent application claims the benefit and priority of Chinese Patent Application No. 202410435183.3 filed with the China National Intellectual Property Administration on Apr. 11, 2024, the disclosure of which is incorporated by reference herein in its entirety as part of the present application.

### TECHNICAL FIELD

The present disclosure belongs to the technical field of functional hydrogels, and relates to a method for preparing a poly(ionic liquid) (PIL)-functionalized cellulose conductive hydrogel.

### BACKGROUND

In recent years, with the development of artificial intelligence and new materials, wearable multifunctional hydrogel-based strain sensors based on bionic skin can convert external stimuli (stretching, compression, bending, expansion, or the like) into electrical signals, and exhibit promising application prospects in human health monitoring, electronic skin, human-computer interaction systems, implantable devices, or the like. The multifunctionality and life span of a hydrogel are primarily determined by the overall mechanical properties of the hydrogel, including stretchability, strength, elasticity, toughness, and fatigue resistance. So far, synthetic polymers, such as the most widely-used polyvinyl alcohol (PVA) and polyacrylamide (PAAm), have still remained the preferred polymer backbones for research and commercial applications because they impart excellent mechanical flexibility and strength to hydrogels. However, these traditional petroleum-derived materials are non-renewable and poorly-biodegradable, and the gel preparation process is carbon-intensive. Nevertheless, the development direction of materials is to simplify the processing and reduce the manufacturing cost and carbon footprint. Therefore, the transformation of the most abundant biological polymers on the earth into high-value products is an effective way to allow environmental sustainability.

Cellulose has attracted widespread attention due to its unique characteristics such as renewability, degradability, low cost, and mechanical/thermal stability. Hydrogels are typically prepared using a bottom-up approach, with nanofibrillar cellulose as the matrix and crosslinking or polymerizing with other monomers. For example, Chinese Patent Application No.CN202310822185.3 discloses a cellulose nanofiber/PAAm double-network hydrogel with an elongation at break of up to 1350% and a breaking strength of up to 290 kPa. Further, the incorporation of monovalent metal salts into the gel network endows it with electric conductivity, thereby meeting the requirements for applications in sensor devices. However, such a hydrogel has a low nanofiber content (which is typically less than 10%), and still cannot meet the growing demand for eco-friendly soft materials globally. In contrast, a functional hydrogel with a high cellulose content can be obtained by dissolving cellulose in an alkali/urea solution and then conducting a phase transformation in a regeneration solution. However, due to the inherent backbone rigidity and crystalline structure of cellulose, cellulose hydrogels prepared by the dissolution-regeneration method exhibit limited strength and stretchability (elongation at break: lower than 100%, strength: lower than 1 MPa, and toughness: about 500 kJ m⁻³), thus restricting the practical applicability of the cellulose hydrogels. As a result, researchers have explored a variety of strategies, including the design of hard and soft phase structures, the introduction of sacrificial bonds, double networks, or reversible interactions, to construct hydrogels with excellent comprehensive mechanical properties. In addition, in order to allow the multi-functional application of a hydrogel-based sensor, in addition to the essential mechanical strength, electric conductivity, and sensing performance, it is necessary to integrate multifunctionality, including self-healing, freezing resistance, antibacterial property, biocompatibility, and water retention, thereby extending the service life of the hydrogel-based sensor and ensuring the stability of the hydrogel-based sensor in a complex environment. However, multifunctionality is often accompanied by processing complexity, such as long preparation time, high monomer selectivity, demand of additional energy, or complicated steps. Therefore, how to prepare a cellulose hydrogel with high mechanical strength, electric conductivity, and multifunctionality by an efficient and simple one-step method is still an urgent problem to be solved.

### SUMMARY

An object of the present disclosure is to provide a method for preparing a PIL-functionalized cellulose conductive hydrogel. The method can lead to a hydrogel material with high mechanical strength, strong electric conductivity, and multifunctionality.

The present disclosure provides the following technical solutions:

A method for preparing a PIL-functionalized cellulose conductive hydrogel, including the following steps:
step 1, preparing an ionic liquid monomer A and an ionic liquid monomer P;
step 2, preparing a PIL: P(Aₓ-co-P_{y}) from the ionic liquid monomer A and the ionic liquid monomer P obtained in step 1; and
step 3, preparing the PIL-functionalized cellulose conductive hydrogel from the PIL obtained in step 2.

In some embodiments, in step 1, the ionic liquid monomer A is prepared by a process including:
adding 1.002 g to 9.015 g of dimethylaminoethyl acrylate (DMAEA) and 1.168 g to 10.515 g of 1-bromohexane (BH) to 5 mL to 45 mL of an acetonitrile solution, and conducting first continuous stirring and a first ultrasonic treatment until a first resulting mixture is dissolved to obtain a solution B;
subjecting the solution B to a first reaction at a temperature of 50° C. to 75° C. for 12 h to 24 h under nitrogen protection to obtain a mixture C;
adding 45 mL to 135 mL of ethyl ether in three batches to the mixture C until a white precipitate is produced; and
vacuum-drying the white precipitate to obtain the ionic liquid monomer A; and
the ionic liquid monomer P is prepared by a process including:
dissolving 90.4 μL to 542.4 μL of 1-vinylimidazole (VI) and 0.2149 g to 1.0742 g of 4-(bromomethyl)phenylboronic acid (BPBA) in 7.5 mL to 37.5 mL of ethyl acetate, and conducting second continuous stirring and a second ultrasonic treatment until a second resulting mixture is dissolved to obtain a solution D;
subjecting the solution D to a second reaction at a temperature of 50° C. to 75° C. for 12 h to 24 h under nitrogen protection to obtain a mixture E;
adding 22.5 mL to 112.5 mL of ethyl acetate in three batches to the mixture E, and conducting rotary evaporation at 35° C. until the ethyl acetate is evaporated to obtain a product F; and
vacuum-drying the product F to obtain the ionic liquid monomer P.

In some embodiments, the step 2 comprises:
adding 0.925 g to 1.079 g of the ionic liquid monomer A, 0.102 g to 0.463 g of the ionic liquid monomer P, and 0.052 g to 0.077 g of azobisisobutyronitrile (AIBN) to a round-bottomed flask, then adding 3.53 mL to 3.81 mL of N,N-dimethylformamide (DMF) to the round-bottomed flask, and conducting ultrasonic mixing to obtain a solution F;
subjecting the solution F to oxygen removal with nitrogen for 10 min to 30 min and then to a third reaction in an oil bath at a temperature of 50° C. to 70° C. for 2 h to 12 h to obtain a mixture G;
adding 1 mL to 15 mL of acetone in three batches to the mixture G to obtain a yellowish precipitate H; and
purifying the yellowish precipitate H by dialysis with water to obtain a purified product, and drying the purified product by a lyophilizer to obtain the P(Aₓ-co-P_{y}).

In some embodiments, the step 3 comprises:
step 3.1, preparing a cellulose solution; and
step 3.2, preparing the PIL-functionalized cellulose conductive hydrogel from the PIL obtained in step 2 and the cellulose solution obtained in step 3.1.

In some embodiments, the step 3.1 comprises:
adding 6 g to 10 g of NaOH and 9 g to 13 g of urea to 77 mL to 85 mL of deionized water, and conducting stirring and mixing to obtain a mixed solution I;
pre-cooling the mixed solution I to obtain a solution I; and
adding 2 g to 5 g of cellulose to the solution I, and stirring until the cellulose is dissolved to obtain a solution J.

In some embodiments, the step 3.2 comprises:
adding 0 g to 0.465 g of the P(Aₓ-co-P_{y}) and 0.1 mL to 1 mL of epichlorohydrin to the solution J obtained in step 3.1, and conducting stirring to obtain a solution K; and
pouring the solution K into a polytetrafluoroethylene (PTFE) mold, subjecting the solution K to standing for 24 h, then to solvent replacement with deionized water for 12 h to obtain the PIL-functionalized cellulose conductive hydrogel denoted as Cell-P(Aₓ-co-P_{y}).

Some embodiments of the present disclosure have the following beneficial effects:

### 1. Excellent Balance Between Mechanical Properties and Electric Conductivity:

In the hydrogel, the dynamic boronic ester bonding is formed between the P(Aₓ-co-P_{y}) and a cis-diol of a cellulose chain, and physical interactions are produced, including node interconnection, a variety of intermolecular and intramolecular hydrogen bonds, and ion-dipole interactions, thereby achieving the excellent balance between mechanical properties and conductivity. In addition, the hydrogel with a rigid cellulose backbone as hard phase not only improves the mechanical strength, but also improves the flexibility by introducing relatively-soft PIL chains. The PIL chains surround or are filled in the rigid network space of cellulose like “bands” or “glues”. Therefore, the soft and hard phase structures provide excellent toughness for the hydrogel. A mechanical tensile test shows that the hydrogel has a maximum compressive modulus of 9.46±0.23 MPa, a maximum tensile strength of 4.30 MPa, an elongation at break of 214.3%, and toughness of 3.64±0.12 MJ m⁻³. A four-probe test shows that the hydrogel has a maximum electric conductivity of 8.82±0.53 mS cm⁻¹.

### 2. Integration of Multifunctionality:

The P(Aₓ-co-P_{y}) combines the design flexibility of ionic liquid monomers and the multifunctional compatibility of a polymer chain, allowing various functional groups to be integrated into the same polymer structure. Therefore, through the functionalization of a cellulose hydrogel with a PIL in an alkali/urea system, a multifunctional cellulose hydrogel with self-healing ability, antibacterial effect, freezing resistance, and long-term stability can be prepared by a simple one-step method.

### 3. Excellent Mechanical Sensing Performance:

The P(Aₓ-co-P_{y}) in the hydrogel as a conductive substance can increase the conductivity and sensing performance. The hydrogel undergoes a significant resistance change and shows strain sensing responsiveness under mechanical stretching, and undergoes a stable resistance change during a 1,000-stretching-cycle loading process, meeting the requirements for the service life and signal stability of sensor devices.

### 4. Promising Application Prospects:

The multifunctional hydrogel-based sensor has all the advantages of the aforementioned hydrogel, and exhibits promising application prospects in human motion monitoring and physiological signal detection. When assembled on the surface of the human skin, the PIL-functionalized cellulose conductive hydrogel-based sensor can monitor the human movement and electrical signals in real time. As the degree of finger bending changes, the hydrogel-based sensor produces a change in resistance responsively and converts the change in resistance into a change in electrical signal.

### 5. Simple Preparation Method:

A PIL is designed and then added to a cellulose solution in an alkali/urea system, such that the multifunctional cellulose conductive hydrogel can be produced in one step, which involves simple operation steps and easy-to-control process parameters. A sensor can be prepared merely by connecting two ends of the cellulose conductive hydrogel to wires and then encapsulating, which is also very simple.

### BRIEF DESCRIPTION OF THE DRAWINGS

FIG. 1A to FIG. 1B show flow charts for preparing an ionic liquid monomer A and an ionic liquid monomer P in the method for preparing a PIL-functionalized cellulose conductive hydrogel according to an embodiment of the present disclosure, respectively, where FIG. 1A is a flow chart for preparing the ionic liquid monomer A, and FIG. 1B is a flow chart for preparing the ionic liquid monomer P;

FIG. 2 is a flow chart for preparing a P(Aₓ-co-P_{y}) in the method for preparing a PIL-functionalized cellulose conductive hydrogel according to an embodiment of the present disclosure;

FIG. 3A to FIG. 3B show a nuclear magnetic resonance spectrum and an infrared spectrum of the ionic liquid monomer A according to an embodiment of the method for preparing a PIL-functionalized cellulose conductive hydrogel in the present disclosure, respectively, where FIG. 3A shows a nuclear magnetic resonance spectrum of the ionic liquid monomer A, and FIG. 3B shows an infrared spectrum of the ionic liquid monomer A;

FIG. 4A to FIG. 4B show a nuclear magnetic resonance spectrum and an infrared spectrum of the ionic liquid monomer P according to an embodiment of the method for preparing a PIL-functionalized cellulose conductive hydrogel in the present disclosure, respectively, where FIG. 4A shows a nuclear magnetic resonance spectrum of the ionic liquid monomer P, and FIG. 4B shows an infrared spectrum of the ionic liquid monomer P;

FIG. 5 shows a nuclear magnetic resonance spectrum of the P(A₈-co-P₂) copolymer according to an embodiment of the method for preparing a PIL-functionalized cellulose conductive hydrogel in the present disclosure;

FIG. 6 shows infrared spectroscopy spectra of the ionic conductive hydrogels and the P(Aₓ-co-P_{y}) copolymers in Examples 1 to 4 of the method for preparing a PIL-functionalized cellulose conductive hydrogel in the present disclosure;

FIG. 7 shows X-ray photoelectron spectroscopy (XPS) spectra of the ionic conductive hydrogels and the P(Aₓ-co-P_{y}) copolymers in Examples 1 to 4 of the method for preparing a PIL-functionalized cellulose conductive hydrogel in the present disclosure;

FIG. 8A to FIG. 8B show mechanical property of the hydrogels in Examples 1 to 4 of a method for preparing a PIL-functionalized cellulose conductive hydrogel in the present disclosure, where FIG. 8A shows tensile stress-strain curves of the PIL-functionalized cellulose conductive hydrogels in Examples 1 to 4, and FIG. 8B shows toughness-compressive modulus graphs of the PIL-functionalized cellulose conductive hydrogels in Examples 1 to 4;

FIG. 9 shows conductivity of the hydrogels in Examples 1 to 4 of the method for preparing a PIL-functionalized cellulose conductive hydrogel in the present disclosure;

FIG. 10A to FIG. 10C show electrochemical impedance spectroscopy (EIS) spectra of the PIL-functionalized cellulose conductive hydrogel prepared in Example 3 of the method for preparing a PIL-functionalized cellulose conductive hydrogel in the present disclosure at different temperatures;

FIG. 11A to FIG. 11C show self-healing performance of the hydrogel prepared in Example 3 of a method for preparing a PIL-functionalized cellulose conductive hydrogel in the present disclosure;

FIG. 12A to FIG. 12B show antibacterial physical images and antibacterial ratio of the PIL-functionalized cellulose conductive hydrogels prepared in Examples 1 to 4 of the method for preparing a PIL-functionalized cellulose conductive hydrogel in the present disclosure, respectively;

FIG. 13A to FIG. 13B show water retention of the hydrogels in Examples 1 and 3 of the method for preparing a PIL-functionalized cellulose conductive hydrogel in the present disclosure;

FIG. 14A to FIG. 14B show sensing performance of the hydrogel-based sensor in Example 3 of the method for preparing a PIL-functionalized cellulose conductive hydrogel in the present disclosure; and

FIG. 15A to FIG. 15B show monitoring signal of the ionic conductive hydrogel-based sensor in Example 3 of the method for preparing a PIL-functionalized cellulose conductive hydrogel in the present disclosure for human finger bending and vocalization behaviors.

### DETAILED DESCRIPTION OF THE EMBODIMENTS

The present disclosure is described in detail below with reference to the accompanying drawings and specific embodiments.

The present disclosure provides a method for preparing a PIL-functionalized cellulose conductive hydrogel, with the design ideas as follows: Firstly, a PIL is designed to endow the cellulose hydrogel with excellent multifunctionality. PIL as a functional macromolecule, the following three main structural designs are considered: (1) reactivity with hydroxyl groups: the phenylboronic acid molecule in a monomer P reacts with the cis-diol of cellulose to form a reversible boronic ester bond; (2) intrinsic ionic conductivity: the structures of an imidazole salt and a quaternary ammonium salt increase the conductivity of the hydrogel, and generate hydrogen bonds and ion-dipole interactions with cellulose; (3) structural adjustability and multifunctional compatibility: the relative flexibility of molecular chains is allowed by structural design, and a variety of functional groups can be integrated into the same structural unit while ensuring excellent water solubility, which allows the customization of material properties according to needs. The PIL macromolecule is directly added to a cellulose solution in an alkali/urea system to prepare the multifunctional cellulose conductive hydrogel of the present disclosure in one step. Through the synergistic effect of multiple interactions (dynamic boronic ester bonding, hydrogen bonding, and ion-dipole interactions) combined with the structural design of soft-hard phases, the hydrogel can exhibit excellent mechanical properties, conductivity, self-healing performance, antibacterial property, biocompatibility, water retention, and sensing performance. In order to allow the above objects, some embodiments of the present disclosure adopt the following materials: DMAEA, BH, acetonitrile, ethyl ether, BPBA, VI, ethyl acetate, AIBN, DMF, acetone, cellulose (cotton linter pulp, DP=600, α-cellulose content: higher than 95%), sodium hydroxide (NaOH), urea, and deionized water.

A method for preparing a PIL-functionalized cellulose conductive hydrogel in some embodiments of the present disclosure specifically includes the following steps:

### Step 1: Preparing an Ionic Liquid Monomer A and an Ionic Liquid Monomer P

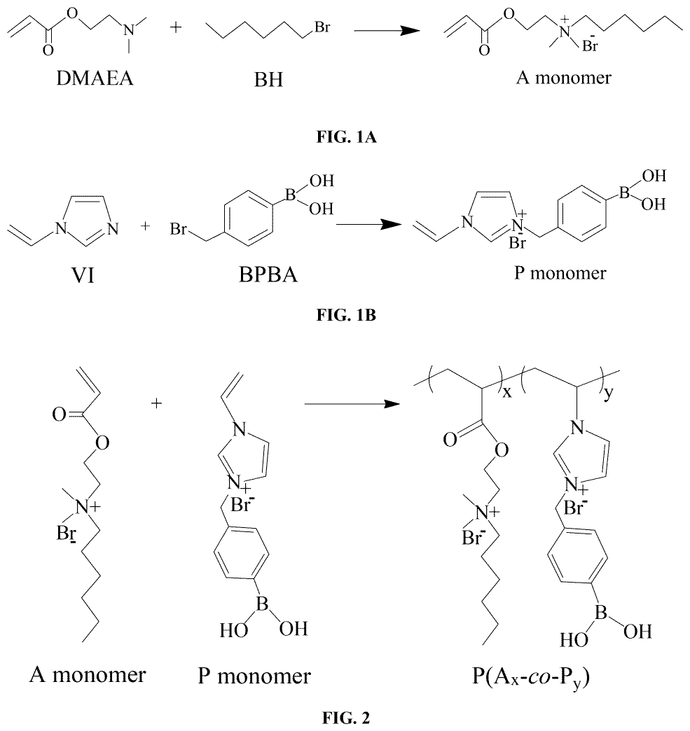

### Step 1.1: Preparing an Ionic Liquid Monomer A as Shown in FIG. 1a:

1.002 g to 9.015 g of DMAEA and 1.168 g to 10.515 g of BH are added to 5 mL to 45 mL of an acetonitrile solution, and continuous stirring and an ultrasonic treatment are conducted until a resulting mixture is completely dissolved to obtain a solution B.

The solution B is subjected to a reaction at a temperature of 50° C. to 75° C. for 12 h to 24 h under nitrogen protection to obtain a mixture C.

45 mL to 135 mL of ethyl ether is added in three batches to the mixture C until a white precipitate is produced.

The white precipitate is vacuum-dried at 45° C. to obtain the ionic liquid monomer A.

### Step 1.2: Preparing an Ionic Liquid Monomer P as Shown in FIG. 1B:

90.4 μL to 542.4 μL of VI and 0.2149 g to 1.0742 g of BPBA are dissolved in 7.5 mL to 37.5 mL of ethyl acetate, and continuous stirring and an ultrasonic treatment are conducted until a resulting mixture is completely dissolved to obtain a solution D.

The solution D is subjected to a reaction at a temperature of 50° C. to 75° C. for 12 h to 24 h under nitrogen protection to obtain a mixture E.

22.5 mL to 112.5 mL of ethyl acetate is added in three batches to the mixture E, and rotary evaporation is conducted at 35° C. until the ethyl acetate is completely evaporated to obtain a product F.

The product F is vacuum-dried at 45° C. to obtain the ionic liquid monomer P.

Step 2: Preparing a P(Aₓ-Co-P_{y}) as Shown in FIG. 2:

0.925 g to 1.079 g of the ionic liquid monomer A, 0.102 g to 0.463 g of the ionic liquid monomer P, and 0.052 g to 0.077 g of AIBN are added to a round-bottomed flask, then 3.53 mL to 3.81 mL of DMF is added to the round-bottomed flask, and ultrasonic mixing is fully conducted to obtain a solution F.

The solution F is subjected to oxygen removal with nitrogen for 10 min to 30 min and then to a reaction in an oil bath at a temperature of 50° C. to 70° C. for 2 h to 12 h to obtain a mixture G.

1 mL to 15 mL of acetone is added in three batches to the mixture G to obtain a yellowish precipitate H. The yellowish precipitate H is purified by dialysis (molecular weight cutoff: 1,000 Da) with water, and then dried by a lyophilizer to obtain the P(Aₓ-co-P_{y}).

### Step 3: Preparing a PIL-Functionalized Cellulose Conductive Hydrogel

### Step 3.1: Preparing a Cellulose Solution:

6 g to 10 g of NaOH and 9 g to 13 g of urea are added to 77 mL to 85 mL of deionized water, and thorough stirring is conducted to obtain a mixed solution I.

The mixed solution I is pre-cooled at−12.5° C. to obtain a solution I.

2 g to 5 g of cellulose is added to the solution I, and stirring is conducted until the cellulose is completely dissolved to obtain a solution J.

Step 3.2: Preparing the PIL-Functionalized Cellulose Conductive Hydrogel from the PIL Obtained in Step 2 and the Solution J Obtained in Step 3.1:

0 g to 0.465 g of the P(Aₓ-co-P_{y}) and 0.1 mL to 1 mL of epichlorohydrin are added to the solution J obtained in step 3.1, and thorough stirring is conducted to obtain a solution K.

The solution K is poured into a PTFE mold, and the solution K is subjected to standing for 24 h, and then to solvent replacement with deionized water for 12 h to obtain the PIL-functionalized cellulose conductive hydrogel denoted as Cell-P(Aₓ-co-P_{y}), such that the preparation of the PIL-functionalized cellulose conductive hydrogel is completed.

The present disclosure provides use of the PIL-functionalized cellulose hydrogel as described in the above technical solution in a sensor. In some embodiments of the present disclosure, the sensor is prepared as follows:

Two ends of the PIL-functionalized cellulose conductive hydrogel are connected to wires, and then encapsulation is conducted.

In some embodiments of the present disclosure, the sensor includes, but is not limited to, a human-computer interaction sensor, an electronic skin, or the like. The electronic skin is also known as a novel wearable flexible bionic tactile sensor.

The ionic liquid monomer A adopted in the following examples is prepared by a process as follows:

1) 3.005 g of DMAEA and 3.505 g of BH are added to 15 mL of an acetonitrile solution, and continuous stirring and an ultrasonic treatment are conducted to until a resulting mixture is complete dissolved to obtain a solution B.

2) The solution B is subjected to a reaction at 70° C. for 24 h under nitrogen protection to obtain a mixture C.

3) 75 mL of ethyl ether is added in three batches to the mixture C until a white precipitate is produced. The white precipitate is vacuum-dried at 45° C. to obtain the ionic liquid monomer A.

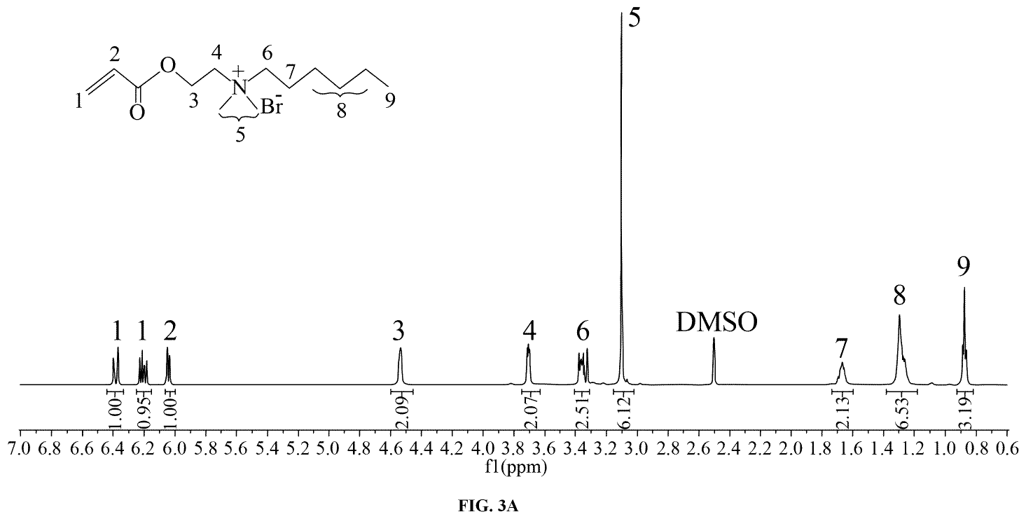

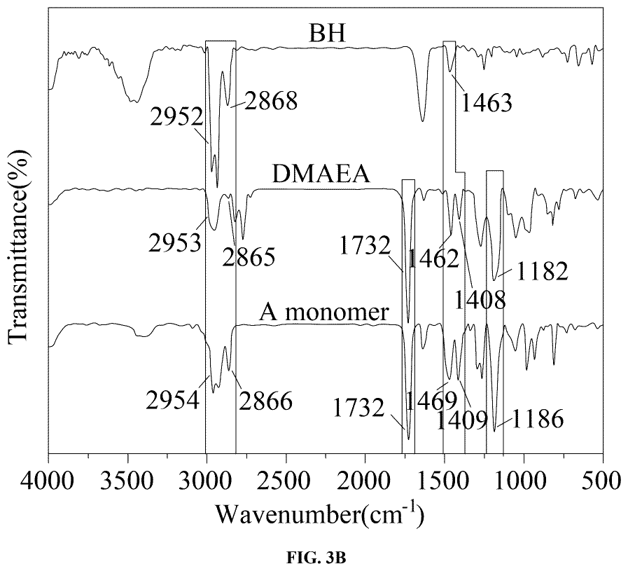

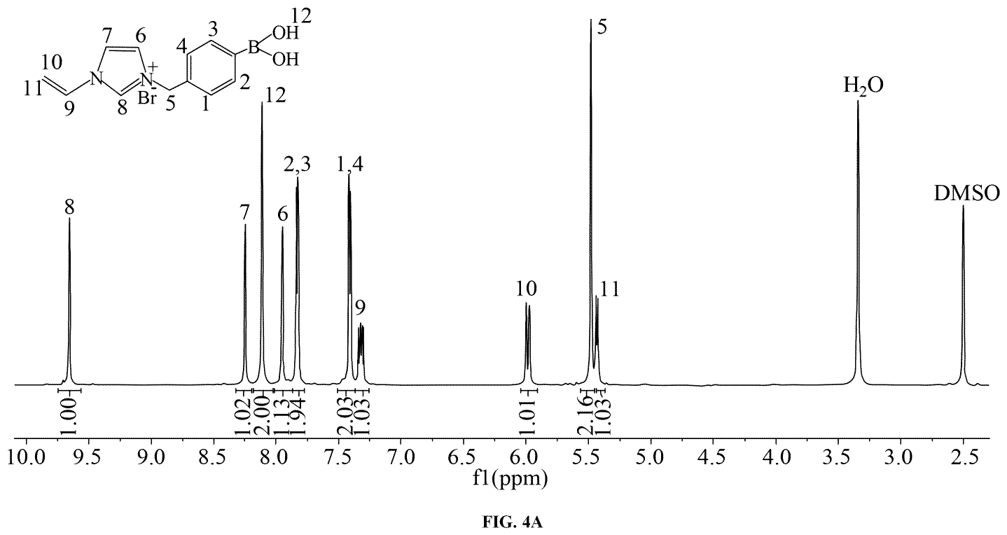

The chemical structure analysis is conducted for the ionic liquid monomer A prepared above by nuclear magnetic resonance spectroscopy and infrared spectroscopy, and the analysis results are shown in FIG. 3A and FIG. 3B. Chemical shifts of different hydrogens can be observed in FIG. 3A:

¹H NMR (600 MHz, DMSO-d6, 25° C., TMS) (ppm): δ=6.38 (dd, 1H, CH, H1), 6.21 (dd, 1H, CH, H1), 6.04 (dd, 1H, CH, H2), 4.55 (m, 2H, CH₂, H3), 3.71 (dd, 2H, CH₂, H4), 3.41 (m, 2H, CH₂, H6), 3.10 (s, 6H, CH₃, H5), 1.74 (m, 2H, CH₂, H7), 1.28 (dt, 6H, CH₂, H8), 0.88 (t, 3H, CH₃, H9).

As shown in FIG. 3B, BH has characteristic peaks for asymmetric stretching and bending vibrations of —CH₂— at 2,952 cm⁻¹ and 1,463 cm⁻¹ respectively and a characteristic peak for a stretching vibration of —CH— at 2,868 cm⁻¹, and three characteristic peaks at 1,732 cm⁻¹, 1,408 cm⁻¹, and 1,182 cm⁻¹ attribute to C═O, C—N, and C—C—O—C stretching vibrations of DMAEA, respectively. All of the above characteristic peaks can be observed in the ionic liquid monomer A, with some shifts. The above results confirm the successful preparation of the ionic liquid monomer A.

The ionic liquid monomer P adopted in the following examples is prepared by a process as follows:

1) 0.4297 g of BPBA and 226 g of VI are dissolved in 15 mL of ethyl acetate, and continuous stirring and an ultrasonic treatment are conducted until a resulting mixture is completely dissolved to obtain a solution D.

2) The solution D is subjected to a reaction at 70° C. for 20 h under nitrogen protection to obtain a mixture E.

3) 45 mL of ethyl acetate is added in three batches to the mixture E, and rotary evaporation is conducted at 35° C. until the ethyl acetate is completely evaporated to obtain a product F.

The product F is vacuum-dried at 45° C. to obtain the ionic liquid monomer P.

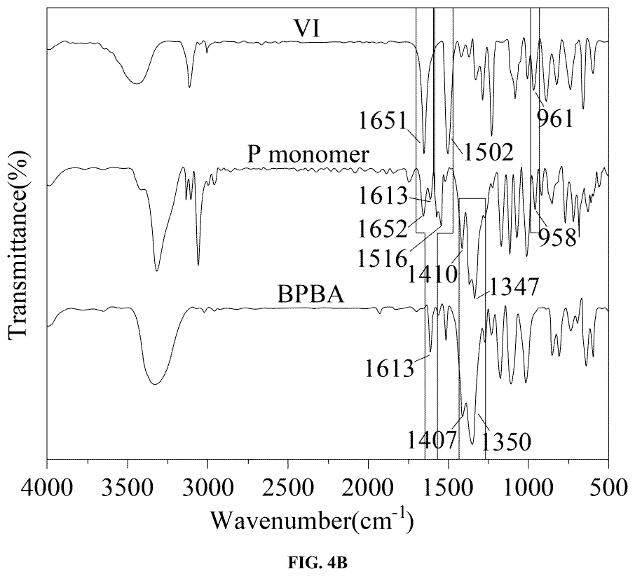

The chemical structure analysis is conducted for the ionic liquid monomer P prepared above by nuclear magnetic resonance spectroscopy and infrared spectroscopy, and the analysis results are shown in FIG. 4A and FIG. 4B. Chemical shifts of different hydrogens can be observed in FIG. 4A:

¹H NMR (600 MHz, DMSO-d6, 25° C., TMS) (ppm): δ=9.65 (s, 1H, CH, H8), 8.25 (s, 1H, CH, H7), 8.11 (s, 2H, OH, H12), 7.95 (s, 1H, CH, H6), 7.83 (d, 2H, CH, H2,3), 7.41 (d, 2H, CH H1, 4), 7.32 (dd, 1H, CH, H9), 5.98 (dd, 1H, CH, H10), 5.48 (s, 2H, CH₂, H5), 5.43 (dd, 1H, CH, H11).

As shown in FIG. 4B, main characteristic peaks of VI at 961 cm⁻¹ (═C—H), 1,502 cm⁻¹ (C—N), and 1,651 cm⁻¹ (C═N) attribute to stretching vibrations of an imidazole ring, respectively, and characteristic peaks of BPBA at 1,350 cm⁻¹ (B—O), 1,407 cm⁻¹ (C—B), and 1,613 cm⁻¹ attribute to vibrations of an aromatic ring backbone. All of the above characteristic peaks can be observed in the ionic liquid monomer P, with some shifts. The above results confirm the successful preparation of the ionic liquid monomer P.

The P(Aₓ-co-P_{y}) adopted in the following examples is prepared by a process as follows:

1.233 g of the ionic liquid monomer A, 0.309 g of the ionic liquid monomer P, and 0.077 g of the AIBN are added to a round-bottomed flask, then 3.81 mL of DMF is added to the round-bottomed flask, and ultrasonic mixing is fully conducted to obtain a solution F.

The solution F is subjected to oxygen removal with nitrogen for 30 min and then to a reaction in an oil bath at 70° C. for 12 h to obtain a mixture G.

15 mL of acetone is added in three batches to the mixture G to obtain a yellowish precipitate H. The yellowish precipitate H is purified by dialysis (molecular weight cutoff: 1,000 Da) with water, and dried by a lyophilizer to obtain the P(A₈-co-P₂).

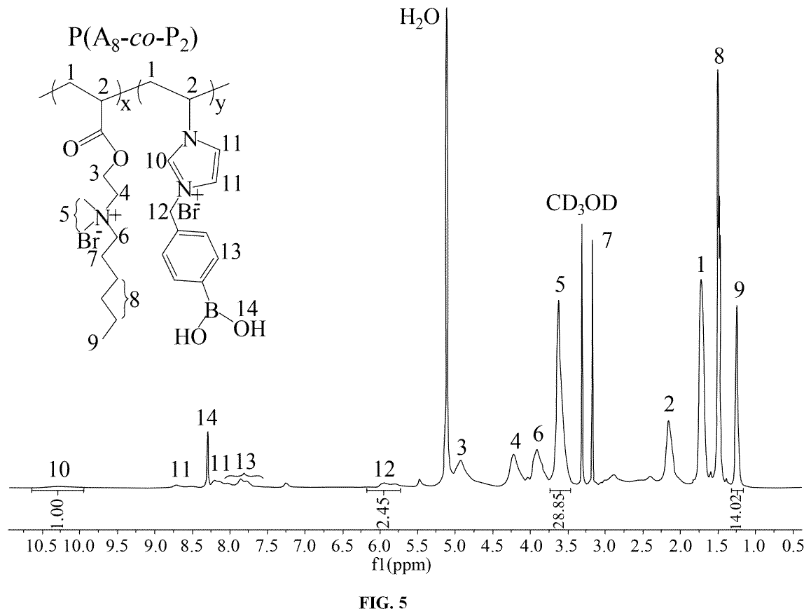

The chemical structure analysis is conducted for the P(A₈-co-P₂) prepared above by nuclear magnetic resonance spectroscopy, and the analysis results are shown in FIG. 5. Chemical shifts of different hydrogens can be observed in FIG. 5. Chemical shift areas of characteristic peaks can be integrated to calculate actual x(A)/y(P)=(area(5+9)/9)/(area(10+12)/3)=8:1.95, which is close to the theoretical ratio of 8:2.

### Example 1

A method for preparing a PIL-functionalized cellulose conductive hydrogel was performed as follows:

### Step 1: Preparing an Ionic Liquid Monomer A and an Ionic Liquid Monomer P

### Step 1.1: Preparing an Ionic Liquid Monomer A:

3.005 g of DMAEA and 3.505 g of BH were added to 15 mL of an acetonitrile solution, and continuous stirring and an ultrasonic treatment were conducted until a resulting mixture was completely dissolved to obtain a solution B. The solution B was subjected to a reaction at 70° C. for 24 h under nitrogen protection to obtain a mixture C. 75 mL of ethyl ether was added in three batches to the mixture C until a white precipitate was produced. The white precipitate was vacuum-dried at 45° C. to obtain the ionic liquid monomer A.

### Step 1.2: Preparing an Ionic Liquid Monomer P:

0.4297 g of BPBA and 226 g of VI were dissolved in 15 mL of ethyl acetate, and continuous stirring and an ultrasonic treatment were conducted until a resulting mixture was completely dissolved to obtain a solution D. The solution D was subjected to a reaction at 70° C. for 20 h under nitrogen protection to obtain a mixture E. 45 mL of ethyl acetate was added in three batches to the mixture E, and rotary evaporation was conducted at 35° C. until the ethyl acetate was completely evaporated to obtain a product F. The product F was vacuum-dried at 45° C. to obtain the ionic liquid monomer P.

Step 2: Preparing a P(A₈-Co-P₂):

1.233 g of the ionic liquid monomer A, 0.309 g of the ionic liquid monomer P, and 0.077 g of AIBN were added to a round-bottomed flask, then 3.81 mL of DMF was added to the round-bottomed flask, and ultrasonic mixing was fully conducted to obtain a solution F. The solution F was subjected to oxygen removal with nitrogen for 30 min and then to a reaction in an oil bath at 70° C. for 12 h to obtain a mixture G. 15 mL of acetone was added in three batches to the mixture G to obtain a yellowish precipitate H. The yellowish precipitate H was purified by dialysis (molecular weight cutoff: 1,000 Da) with water, and dried by a lyophilizer to obtain the P(A₈-co-P₂).

### Step 3: Preparing a PIL-Functionalized Cellulose Conductive Hydrogel

### Step 3.1: Preparing a Cellulose Solution:

7 g of NaOH and 12 g of urea were added to 81 mL of deionized water, and thorough stirring was conducted to obtain a mixed solution I. The mixed solution I was pre-cooled at−12.5° C. to obtain a solution I. 3.1 g of cellulose was added to the solution I, and stirring was conducted until the cellulose was completely dissolved to obtain a solution J.

Step 3.2: Preparing a Hydrogel from the PIL Obtained in Step 2 and the Solution J Obtained In Step 3.1:

0 g of the P(A₈-co-P₂) and 0.1 mL of epichlorohydrin were added to the solution J obtained in step 3.1, and thorough stirring was conducted to obtain a solution K. The solution K was poured into a PTFE mold, the solution K was subjected to standing for 24 h, and then to solvent replacement with deionized water for 12 h to obtain a pure cellulose hydrogel denoted as Cell, such that the preparation of the cellulose hydrogel was completed.

Two ends of the hydrogel were connected to wires and then encapsulation was conducted to obtain a hydrogel-based sensor.

### Example 2

A method for preparing a PIL-functionalized cellulose conductive hydrogel was performed as follows:

### Step 1: Preparing an Ionic Liquid Monomer A and an Ionic Liquid Monomer P

### Step 1.1: Preparing an Ionic Liquid Monomer A:

3.005 g of DMAEA and 3.505 g of BH were added to 15 mL of an acetonitrile solution, and continuous stirring and an ultrasonic treatment were conducted until a resulting mixture was completely dissolved to obtain a solution B. The solution B was subjected to a reaction at 70° C. for 24 h under nitrogen protection to obtain a mixture C. 75 mL of ethyl ether was added in three batches to the mixture C until a white precipitate was produced. The white precipitate was vacuum-dried at 45° C. to obtain the ionic liquid monomer A.

### Step 1.2: Preparing an Ionic Liquid Monomer P:

0.4297 g of BPBA and 226 g of VI were dissolved in 15 mL of ethyl acetate, and continuous stirring and an ultrasonic treatment were conducted until a resulting mixture was completely dissolved to obtain a solution D. The solution D was subjected to a reaction at 70° C. for 20 h under nitrogen protection to obtain a mixture E. 45 mL of ethyl acetate was added in three batches to the mixture E, and rotary evaporation was conducted at 35° C. until the ethyl acetate was completely evaporated to obtain a product F. The product F was vacuum-dried at 45° C. to obtain the ionic liquid monomer P.

Step 2: Preparing a P(A₈-Co-P₂):

1.233 g of the ionic liquid monomer A, 0.309 g of the ionic liquid monomer P, and 0.077 g of AIBN were added to a round-bottomed flask, then 3.81 mL of DMF was added to the round-bottomed flask, and ultrasonic mixing was fully conducted to obtain a solution F. The solution F was subjected to oxygen removal with nitrogen for 30 min and then to a reaction in an oil bath at 70° C. for 12 h to obtain a mixture G. 15 mL of acetone was added in three batches to the mixture G to obtain a yellowish precipitate H. The yellowish precipitate H was purified by dialysis (molecular weight cutoff: 1,000 Da) with water, and dried by a lyophilizer to obtain the P(A₈-co-P₂).

### Step 3: Preparing a PIL-Functionalized Cellulose Conductive Hydrogel

### Step 3.1: Preparing a Cellulose Solution:

7 g of NaOH and 12 g of urea were added to 81 mL of deionized water, and thorough stirring was conducted to obtain a mixed solution I. The mixed solution I was pre-cooled at−12.5° C. to obtain a solution I. 3.1 g of cellulose was added to the solution I, and stirring was conducted until the cellulose was completely dissolved to obtain a solution J.

Step 3.2: Preparing a Hydrogel from the PIL Obtained in Step 2 and the Solution J Obtained In Step 3.1:

0.155 g of the P(A₈-co-P₂) and 0.1 mL of epichlorohydrin were added to the solution J obtained in step 3.1, and thorough stirring was conducted to obtain a solution K. The solution K was poured into a PTFE mold, the solution K was subjected to standing for 24 h, and then to solvent replacement with deionized water for 12 h to obtain the PIL-functionalized cellulose conductive hydrogel denoted as Cell-P(A₈-co-P₂)5%, such that the preparation of the PIL-functionalized cellulose conductive hydrogel was completed.

Two ends of the hydrogel were connected to wires and then encapsulation was conducted to obtain a hydrogel-based sensor.

### Example 3

A method for preparing a PIL-functionalized cellulose conductive hydrogel was performed as follows:

### Step 1: Preparing an Ionic Liquid Monomer A and an Ionic Liquid Monomer P

### Step 1.1: Preparing an Ionic Liquid Monomer A:

3.005 g of DMAEA and 3.505 g of BH were added to 15 mL of an acetonitrile solution, and continuous stirring and an ultrasonic treatment were conducted until a resulting mixture was completely dissolved to obtain a solution B. The solution B was subjected to a reaction at 70° C. for 24 h under nitrogen protection to obtain a mixture C. 75 mL of ethyl ether was added in three batches to the mixture C until a white precipitate was produced. The white precipitate was vacuum-dried at 45° C. to obtain the ionic liquid monomer A.

### Step 1.2: Preparing an Ionic Liquid Monomer P:

0.4297 g of BPBA and 226 g of VI were dissolved in 15 mL of ethyl acetate, and continuous stirring and an ultrasonic treatment were conducted until a resulting mixture was completely dissolved to obtain a solution D. The solution D was subjected to a reaction at 70° C. for 20 h under nitrogen protection to obtain a mixture E. 45 mL of ethyl acetate was added in three batches to the mixture E, and rotary evaporation was conducted at 35° C. until the ethyl acetate was completely evaporated to obtain a product F. The product F was vacuum-dried at 45° C. to obtain the ionic liquid monomer P.

Step 2: Preparing a P(A₈-Co-P₂):

1.233 g of the ionic liquid monomer A, 0.309 g of the ionic liquid monomer P, and 0.077 g of AIBN were added to a round-bottomed flask, then 3.81 mL of DMF was added to the round-bottomed flask, and ultrasonic mixing was fully conducted to obtain a solution F. The solution F was subjected to oxygen removal with nitrogen for 30 min and then to a reaction in an oil bath at 70° C. for 12 h to obtain a mixture G. 15 mL of acetone was added in three batches to the mixture G to obtain a yellowish precipitate H. The yellowish precipitate H was purified by dialysis (molecular weight cutoff: 1,000 Da) with water, and dried by a lyophilizer to obtain the P(A₈-co-P₂).

### Step 3: Preparing a PIL-Functionalized Cellulose Conductive Hydrogel

### Step 3.1: Preparing a Cellulose Solution:

7 g of NaOH and 12 g of urea were added to 81 mL of deionized water, and thorough stirring was conducted to obtain a mixed solution I. The mixed solution I was pre-cooled at−12.5° C. to obtain a solution I. 3.1 g of cellulose was added to the solution I, and stirring was conducted until the cellulose was completely dissolved to obtain a solution J.

Step 3.2: Preparing a Hydrogel from the PIL Obtained in Step 2 and the Solution J Obtained In Step 3.1:

0.310 g of the P(A₈-co-P₂) and 0.1 mL of epichlorohydrin were added to the solution J obtained in the step 3.1, and thorough stirring was conducted to obtain a solution K. The solution K was poured into a PTFE mold, the solution K was subjected to standing for 24 h, and then to solvent replacement with deionized water for 12 h to obtain the PIL-functionalized cellulose conductive hydrogel denoted as Cell-P(A₈-co-P₂)10%, such that the preparation of the PIL-functionalized cellulose conductive hydrogel was completed.

Two ends of the hydrogel were connected to wires and then encapsulation was conducted to obtain a hydrogel-based sensor.

### Example 4

A method for preparing a PIL-functionalized cellulose conductive hydrogel was performed as follows:

### Step 1: Preparing an Ionic Liquid Monomer A and an Ionic Liquid Monomer P

STEP 1.1: Preparing an ionic liquid monomer A:

3.005 g of DMAEA and 3.505 g of BH were added to 15 mL of an acetonitrile solution, and continuous stirring and an ultrasonic treatment were conducted until a resulting mixture was completely dissolved to obtain a solution B. The solution B was subjected to a reaction at 70° C. for 24 h under nitrogen protection to obtain a mixture C. 75 mL of ethyl ether was added in three batches to the mixture C until a white precipitate was produced. The white precipitate was vacuum-dried at 45° C. to obtain the ionic liquid monomer A.

### Step 1.2: Preparing an Ionic Liquid Monomer P:

0.4297 g of BPBA and 226 g of VI were dissolved in 15 mL of ethyl acetate, and continuous stirring and an ultrasonic treatment were conducted until a resulting mixture was completely dissolved to obtain a solution D. The solution D was subjected to a reaction at 70° C. for 20 h under nitrogen protection to obtain a mixture E. 45 mL of ethyl acetate was added in three batches to the mixture E, and rotary evaporation was conducted at 35° C. until the ethyl acetate was completely evaporated to obtain a product F. The product F was vacuum-dried at 45° C. to obtain the ionic liquid monomer P.

Step 2: Preparing a P(A₈-Co-P₂):

1.233 g of the ionic liquid monomer A, 0.309 g of the ionic liquid monomer P, and 0.077 g of AIBN were added to a round-bottomed flask, then 3.81 mL of DMF was added to the round-bottomed flask, and ultrasonic mixing was fully conducted to obtain a solution F. The solution F was subjected to oxygen removal with nitrogen for 30 min and then to a reaction in an oil bath at 70° C. for 12 h to obtain a mixture G. 15 mL of acetone was added in three batches to the mixture G to obtain a yellowish precipitate H. The yellowish precipitate H was purified by dialysis (molecular weight cutoff: 1,000 Da) with water, and dried by a lyophilizer to obtain the P(A₈-co-P₂).

### Step 3: Preparing a PIL-Functionalized Cellulose Conductive Hydrogel

### Step 3.1: Preparing a Cellulose Solution:

7 g of NaOH and 12 g of urea were added to 81 mL of deionized water, and thorough stirring was conducted to obtain a mixed solution I. The mixed solution I was pre-cooled at−12.5° C. to obtain a solution I. 3.1 g of cellulose was added to the solution I, and stirring was conducted until the cellulose was completely dissolved to obtain a solution J.

Step 3.2: Preparing a Hydrogel from the PIL Obtained in Step 2 and the Solution J Obtained In Step 3.1:

0.465 g of the P(A₈-co-P₂) and 0.1 mL of epichlorohydrin were added to the solution J obtained in step 3.1, and thorough stirring was conducted to obtain a solution K. The solution K was poured into a PTFE mold, the solution K was subjected to standing for 24 h, and then to solvent replacement with deionized water for 12 h to obtain the PIL-functionalized cellulose conductive hydrogel denoted as Cell-P(A₈-co-P₂)15%, such that the preparation of the PIL-functionalized cellulose conductive hydrogel was completed.

Two ends of the hydrogel were connected to wires and then encapsulation was conducted to obtain a hydrogel-based sensor.

The hydrogels and the sensors prepared in Examples 1 to 4 each were subjected to performance analysis as follows:

### (1) Analysis of Chemical Structures

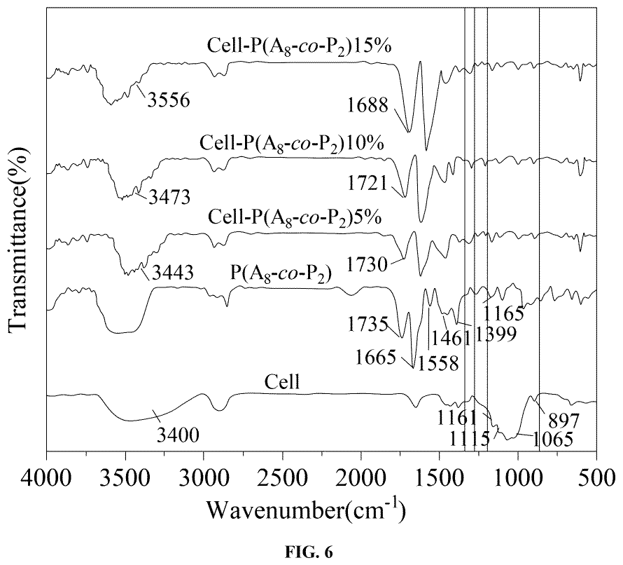

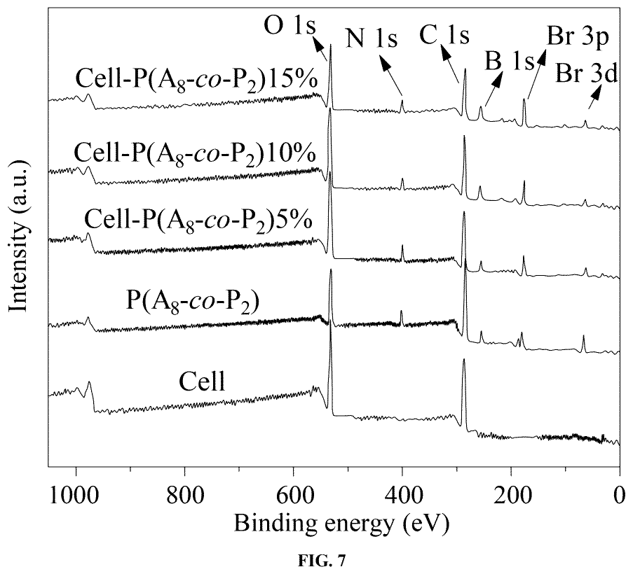

The chemical structure characterization was conducted for the hydrogels prepared in Examples 1 to 4 by infrared spectroscopy and XPS, and the characterization results are shown in FIG. 6 and FIG. 7. As shown in FIG. 6, in a Fourier transform infrared spectroscopy spectrum of the Cell, the wideband around 3,400 cm⁻¹ attributes to the stretching vibration of —OH, and the peak at 2,904 cm⁻¹ attributes to the stretching vibration of a C—H bond. Peaks at 1,161 cm⁻¹ and 897 cm⁻¹ attribute to bending and symmetrical stretching of C—O—C on the β-(1-4) glycosidic bond, respectively. For the P(A₈-co-P₂) sample, characteristic peaks are observed at 1,665 cm⁻¹, 1,558 cm⁻¹, 1,461 cm⁻¹, and 1,399 cm⁻¹, which attribute to the stretching vibration of C═N, C—N, B—O, and C—B in the imidazole moiety, respectively. In addition, peaks at 1,735 cm⁻¹ and 1,165 cm⁻¹ attribute to the stretching vibrations of carbonyl (C═O) and C—C—O—C of the ionic liquid monomer A, respectively. For the Cell-P(A₈-co-P₂) hydrogel, intensities of peaks of C═O, C═N, and C—N stretching vibrations of P(A₈-co-P₂) at 1,735 cm⁻¹, 1,665 cm⁻¹, and 1,558 cm⁻¹ increase and shift with the increase of the content of P(A₈-co-P₂). Specifically, as the content of P(A₈-co-P₂) increases from 5% to 15%, the peak representing the C═O stretching vibration at 1,730 cm⁻¹ shifts to 1,688 cm⁻¹, and C═N and C—N bands overlap and shift to low wavelengths (1,622 cm⁻¹ to 1,590 cm⁻¹), indicating that C═O, C═N, and C—N groups affect the interaction between cellulose and a PIL molecule. In addition, when the content of a PIL is high, a broad peak of O—H stretching shifts to a high wavenumber (blue shift). As the content of the PIL increases from 5% to 15%, the characteristic peak of the pure cell hydrogel at 3,400 cm⁻¹ (corresponding to an intermolecular hydrogen bond) shifts by 43 cm⁻¹ to 56 cm⁻¹, indicating that the cell and the PIL form an intermolecular interaction through hydrogen bonds, namely, “blue shift”. In addition, the spectra of all hydrogels have the peak of the asymmetrical stretching vibration of B—O—C around 1,298 cm⁻¹, indicating that a boronic ester bond is formed between a PIL and a cis-diol site of a Cell chain in the polymer network.

The elemental composition of the surface of each hydrogel was further analyzed by XPS. As shown in FIG. 7, the XPS spectrum of the Cell hydrogel has only C is and O is peaks, while P(A₈-co-P₂) has N Is, Br 3p, and Br 3d peaks. In contrast, the spectrum of the Cell-P(A₈-co-P₂) hydrogel has O 1s, N 1s, C 1s, B 1s, Br 3p, and Br 3d peaks simultaneously.

### (2) Analysis of Mechanical Properties

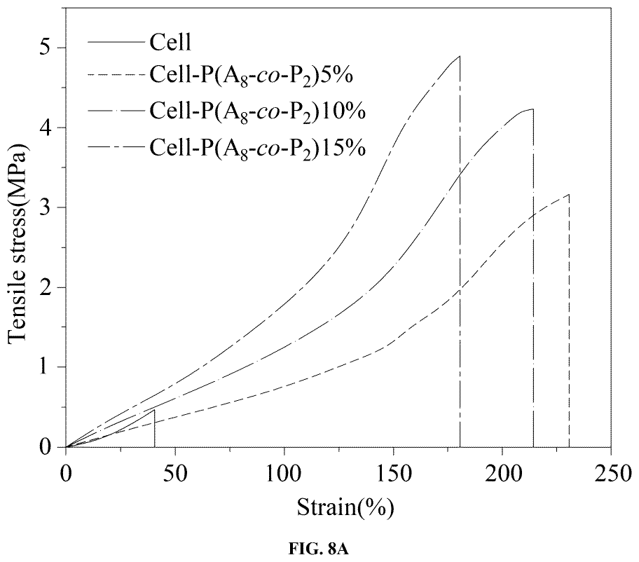

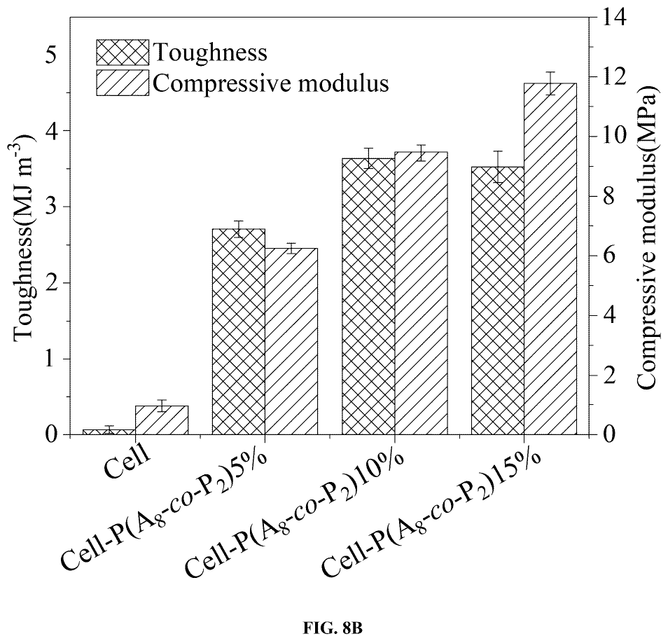

Mechanical properties of the ionic conductive hydrogels prepared in Examples 1 to 4 were tested by a universal testing machine, and the test results are shown in FIG. 8A and FIG. 8B. FIG. 8A shows tensile stress-strain curves of the ionic conductive hydrogels in Examples 1 to 4. FIG. 8B shows toughness-compressive modulus graphs of the ionic conductive hydrogels in Examples 1 to 4. As shown in FIG. 8A, the introduction of P(A₈-co-P₂)5% also improves the stretchability and strength of the Cell hydrogel (a maximum tensile stress and strain at break are 3.16 MPa and 230.4%, respectively, which are 6.9 and 5.7 times that of the pure Cell hydrogel, respectively). As the content of P(A₈-co-P₂) increases from 5% to 15%, the tensile stress increases from 3.16 MPa to 4.92 MPa, while the tensile strain gradually decreases from 230.4% to 181.1%. These results indicate that the fracture stress and rigidity of a hydrogel can be adjusted by increasing intramolecular forces, physical entanglement, and crosslinking density, thereby enhancing the mechanical strength of the hydrogel. In addition, as shown in FIG. 8B, when the content of P(A₈-co-P₂) increases from 0% to 15%, the toughness first increases from 0.07±0.04 MJ m⁻³ to 3.64±0.12 MJ m⁻³ and then decreases slightly to 3.53±0.20 MJ m⁻³. The Cell-P(A₈-co-P₂)10% hydrogel with a small amount of P(A₈-co-P₂) added has a significantly higher compressive modulus than that of the Cell hydrogel (9.8 times higher).

### (3) Analysis of Conductivity and Freezing Resistance

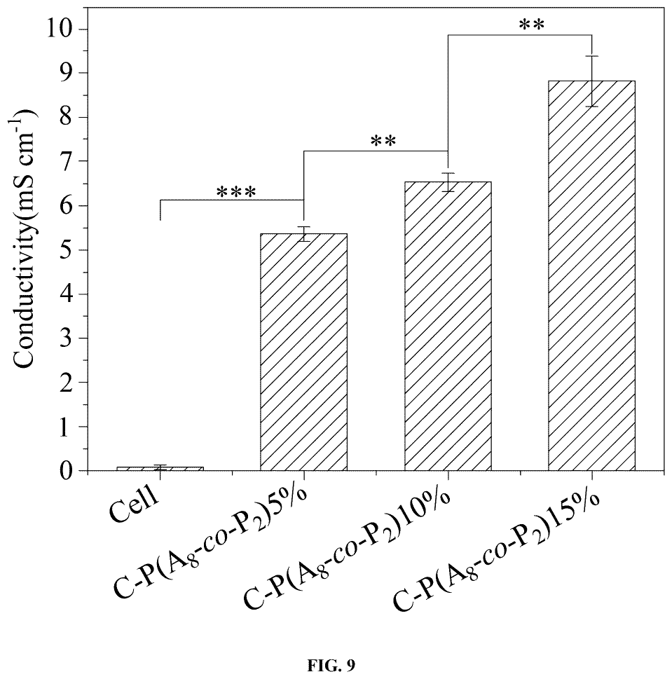

The conductivity of each of the ionic conductive hydrogels prepared in Examples 1 to 4 was tested and evaluated by a four-probe. As shown in FIG. 9, the pure Cell hydrogel is nearly non-conductive due to lack of ionic charge carriers. As the content of P(A₈-co-P₂) increases from 5% to 15%, the corresponding ionic conductivity of the hydrogels also increases monotonically from 5.37±0.13 mS cm⁻¹ to 8.82±0.53 mS cm⁻¹. In FIG. 9, the asterisk (*) indicates statistically-significant difference at the 0.05 level, ^{ns}P>0.05, *P<0.05, **P<0.01, and ***P<0.001.

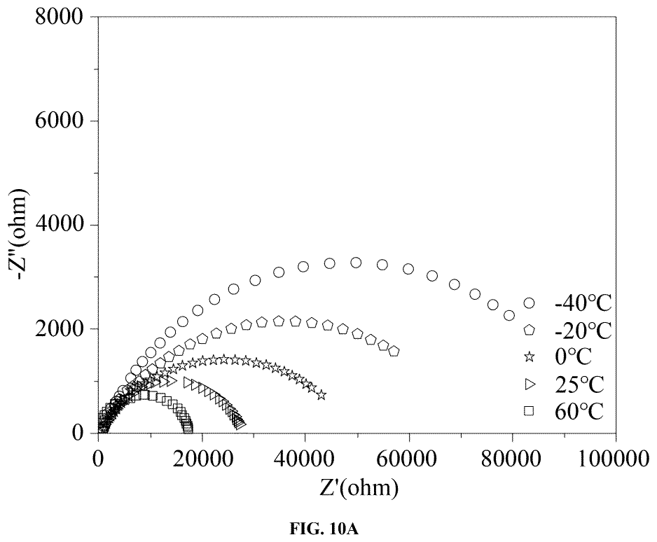

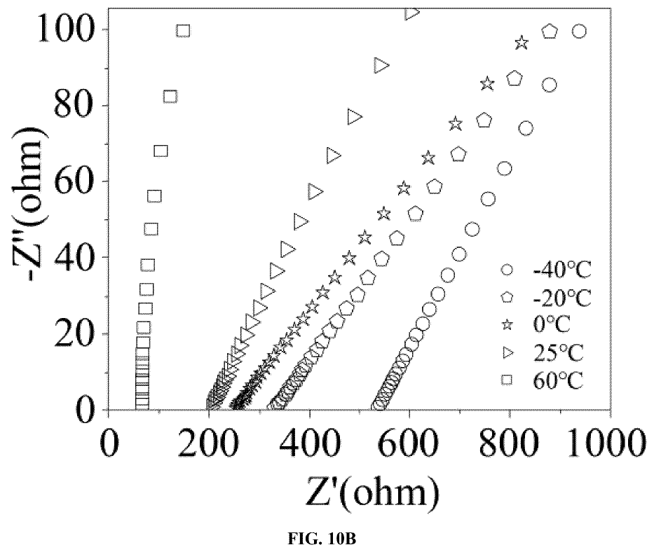

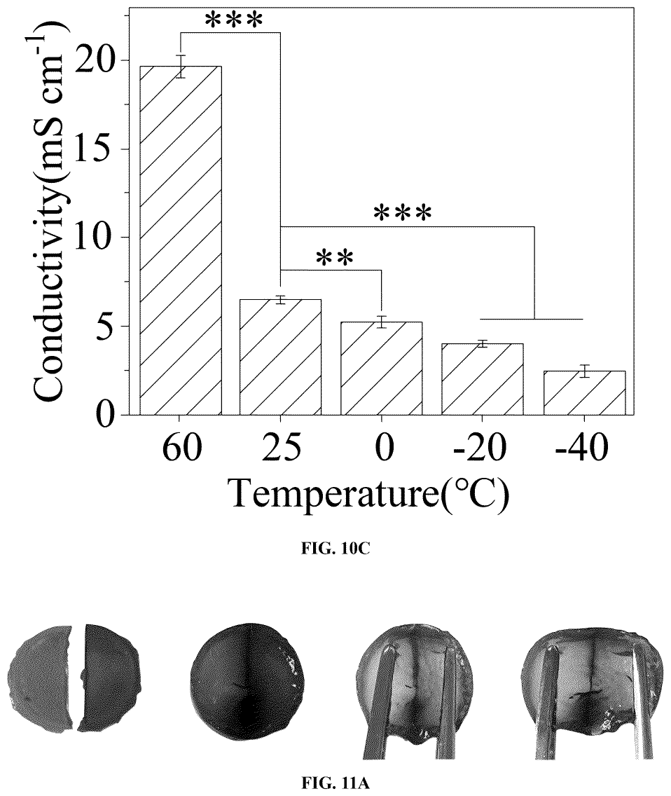

The freezing resistance of the ionic conductive hydrogel prepared in Example 3 was also tested by EIS. As shown in FIG. 10A to FIG. 10C, the lower the temperature, the higher the impedance and the lower the electric conductivity, which is attributed to the fact that the ion mobility is limited at a low temperature. However, even in a −40° C. environment, the ionic conductivity of the Cell-P(A₈-co-P₂)10% hydrogel can still reach 2.48 mS cm⁻¹. This is because water molecules are tightly bonded to PIL groups through a charge-dipole or dipole-dipole interaction, thus lowering the freezing point (freezing-resistant effect).

### (4) Self-Healing Performance

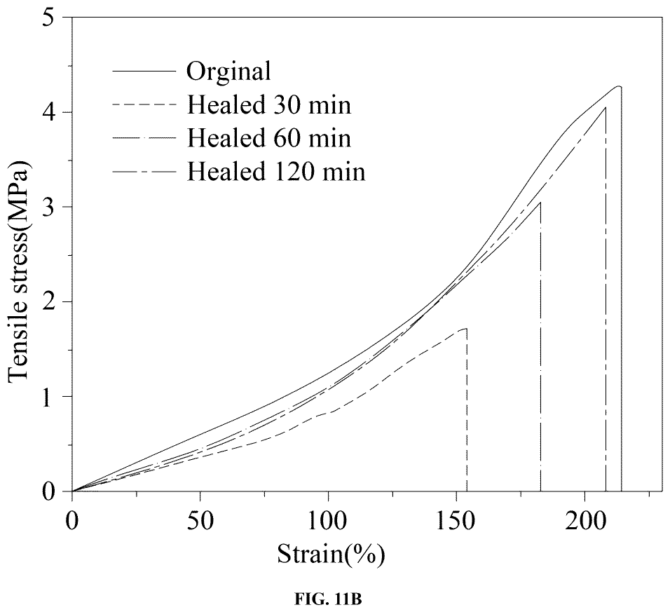

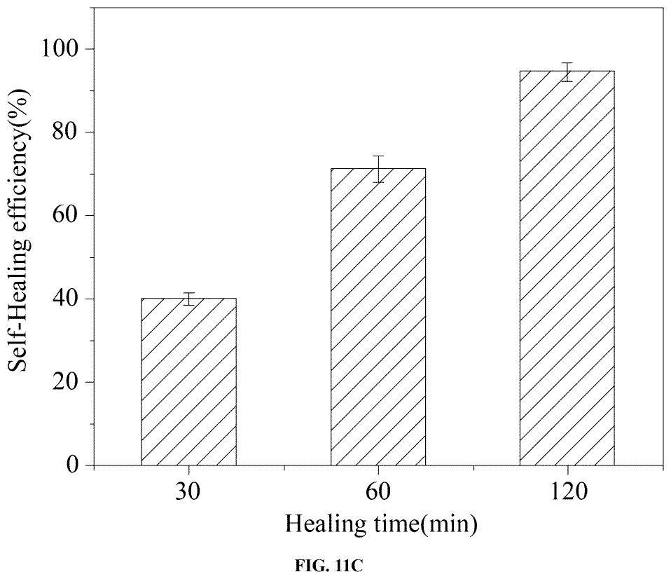

The self-healing performance of the ionic conductive hydrogel prepared in Example 3 was analyzed by observation, an optical microscope, and a universal testing machine. As shown in FIG. 11A (from left to right: images of the hydrogel cut into two half-disc-shaped parts; the two half-disc-shaped parts of the hydrogel in contact for 30 min; the hydrogel after being in contact for 30 min clamped with tweezers showing it could heal into a complete hydrogel; and the hydrogel after self-healing for 30 min stretched with tweezers showing it could withstand tensile force without breaking), after the Cell-P(A₈-co-P₂)10% hydrogel was cut into two half-disc-shaped parts, and the two half-disc-shaped parts of the hydrogel were in contact for 120 min such, the damaged interface reconnected, and the hydrogel could withstand stretching perpendicular to the cut surface without cracking. This is because a boronic ester bond, an ion-dipole interaction, and a hydrogen bond inside the hydrogel can be reconstituted immediately. The tensile strengths and corresponding healing efficiencies (HEs) of the hydrogel at different self-healing times were determined by rheological measurements (FIG. 11B to FIG. 11C). Due to a dynamic crosslinked structure, the Cell-P(A₈-co-P₂)10% hydrogel has excellent self-healing performance, and exhibits a self-healing efficiency of 94.5±2.0% after 150 min of self-healing.

FIG. 11A shows the physical images of the self-healing performance. FIG. 11B shows the stresses-strain diagrams before and after self-healing. FIG. 11C shows the self-healing efficiencies diagram.

### (5) Antibacterial Properties

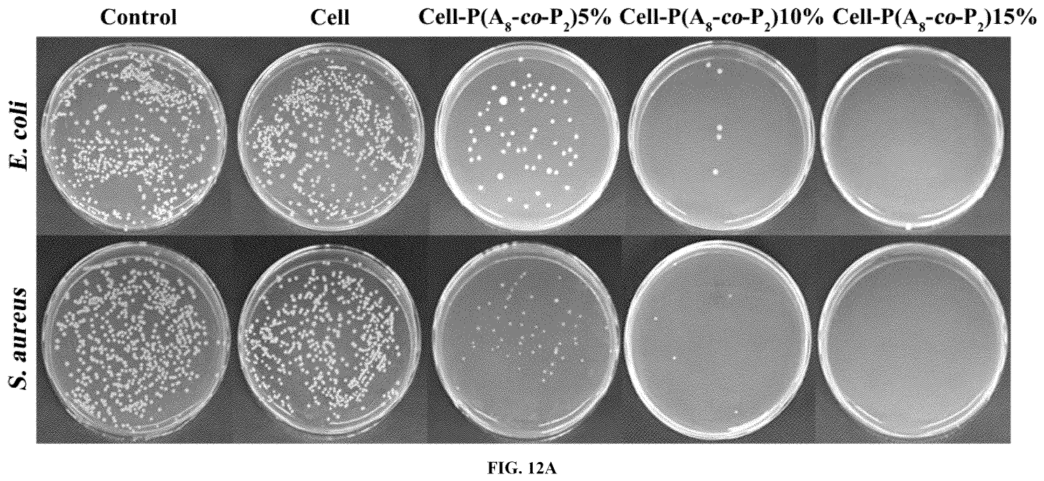

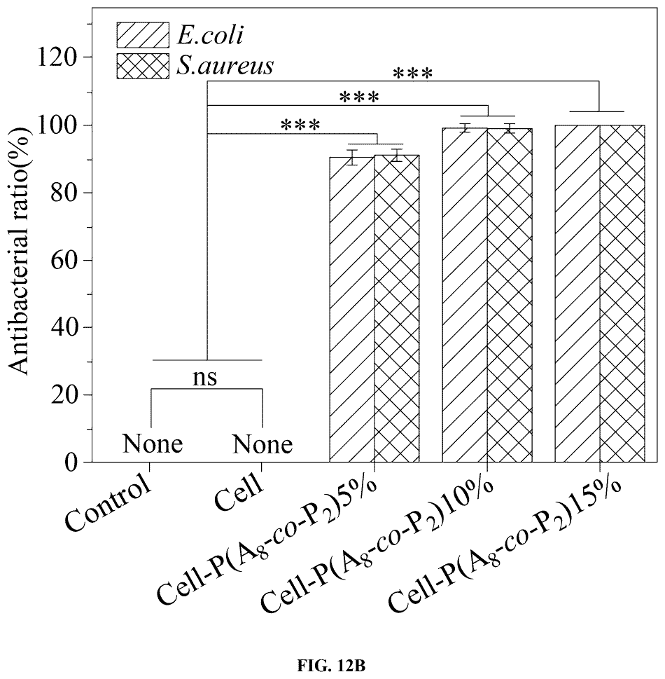

A hydrogel-based multifunctional sensor, especially a sensor that direct contacts with human skin for monitoring health and movement, should prevent a bacterial infection. As a proof of concept, the two representative bacteria of Escherichia coli (E. coli, gram-negative) and Staphylococcus aureus (S. aureus, gram-positive) were adopted to test antibacterial properties of the ionic conductive hydrogels prepared in Examples 1 to 4. As shown in FIG. 12A to FIG. 12B (in FIG. 12A, the first row from left to right shows the antibacterial physical images of a blank control sample and the PIL-functionalized cellulose conductive hydrogels prepared in Examples 1 to 4 against E. coli (gram-negative); and the second row from left to right shows the antibacterial physical images of a blank control sample and the PIL-functionalized cellulose conductive hydrogels prepared in Examples 1 to 4 against S. aureus (gram-positive)), the antibacterial properties of the modified hydrogels are positively correlated with the content of P(A₈-co-P₂). For example, when the content of P(A₈-co-P₂) increases from 5% to 15%, the antibacterial ratios of the Cell-P(A₈-co-P₂) hydrogel against E. coli and S. aureus increase from 90.6±1.98% and 91.2±1.57% to 100%, respectively. This is because there is an electrostatic interaction between a cation of an ionic liquid monomer A as a quaternary ammonium salt antibacterial agent in the PIL and a negatively-charged cell membrane, and a long hydrophobic alkyl segment is introduced to destroy the bacterial cell membrane. This superior antibacterial ability meets the antibacterial requirements for electronic devices attached to the skin. FIG. 12A shows the antibacterial physical images. FIG. 12B shows the antibacterial ratios graph.

### (6) Long-Term Stability

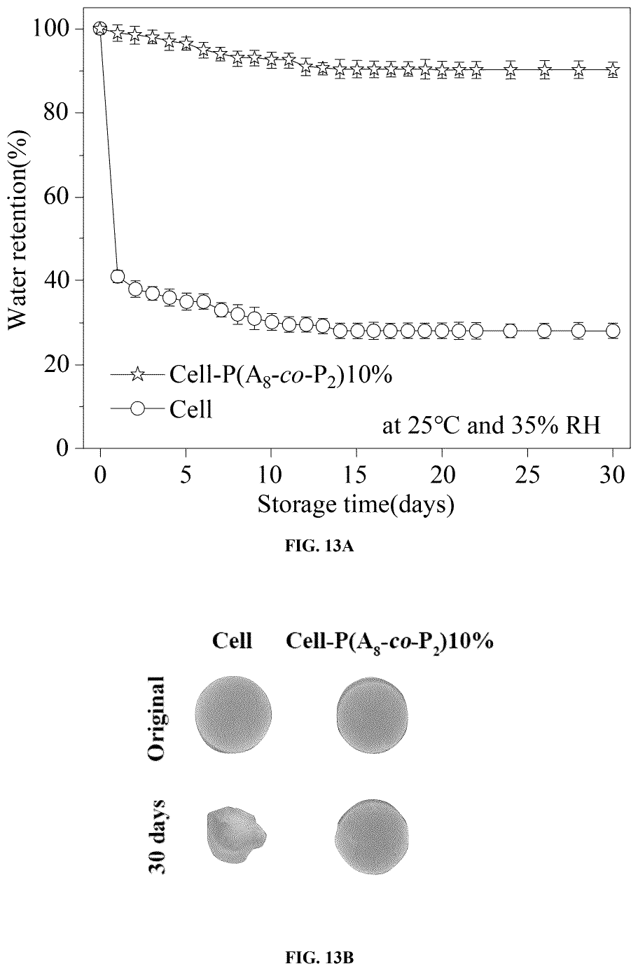

Most hydrogels are prone to dehydration, which often leads to degradation or even loss of properties of a material. Therefore, the long-term stability of each of the ionic conductive hydrogels prepared in Examples 1 and 3 was verified by evaluating their visual appearance, water retention, mechanical properties, ionic conductivity, and self-healing performance. As shown in FIG. 13A to FIG. 13B, after the pure Cell hydrogel was stored at 25° C. and 35% relative humidity (RH) for 30 days, the pure Cell hydrogel lost 72.1±1.6% of its original weight due to water evaporation, becoming an inelastic shrunken dry gel. In contrast, the appearance change of the Cell-P(A₈-co-P₂)10% hydrogel was negligible, and its weight still remained at 90.2±1.7% of the initial weight after 30 days of storage.

### (7) Analysis of Mechanical Sensing Performance

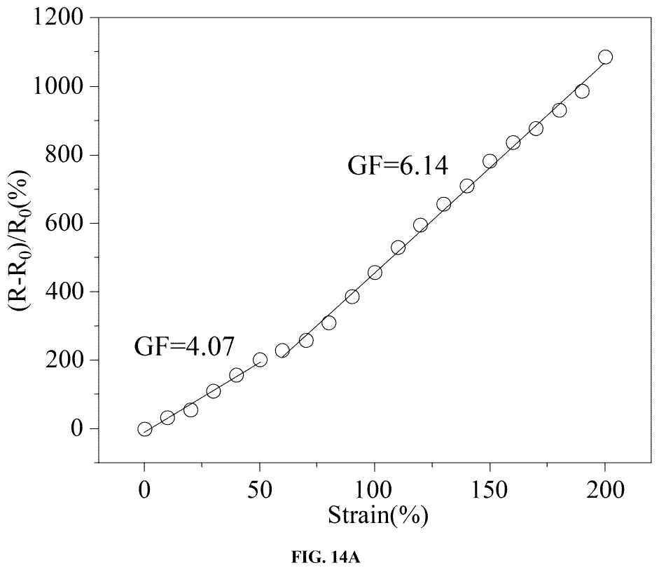

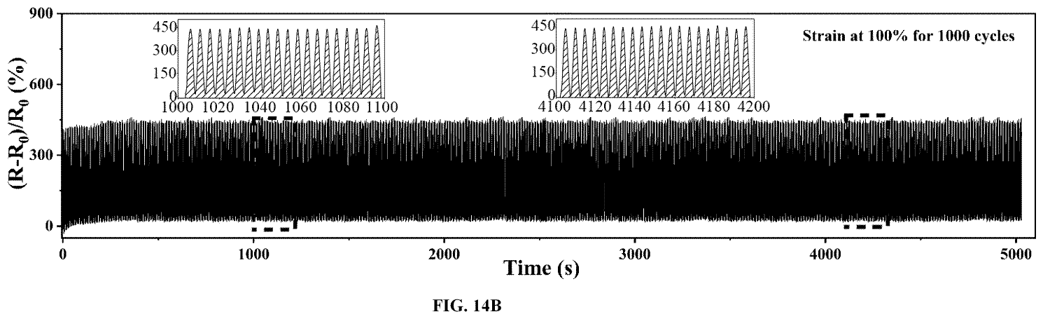

The mechanical sensing performance of the ionic conductive hydrogel-based sensor prepared in Example 3 was tested by a four-probe, a digital electric bridge, and a cyclic reciprocating slide rail. The test results are shown in FIG. 14A and FIG. 14B. As shown in FIG. 14A, the (R—R₀)/R₀ value increases with the increase of strain, and the sensitivity (GF) of the hydrogel is divided into two linear response regions: 0% to 50% region with the GF of 4.07 and 50% to 200% region with the GF of 6.14, indicating that the sensor has high sensitivity. As shown in FIG. 14B, the Cell-P(A₈-co-P₂)10% hydrogel can undergo 1,000 times continuously cycling under 100% strain, maintaining excellent amplitude and waveform information with almost no obvious attenuation in the resistance change signal, proving its signal reliability. FIG. 14A shows the sensitivity of the ionic conductive hydrogel-based sensor in Example 3. FIG. 14B shows that the ionic conductive hydrogel-based sensor in Example 3 can cycle 1,000 times under 100% strain.

### (8) Analysis of Real-Time Sensing on the Human Body

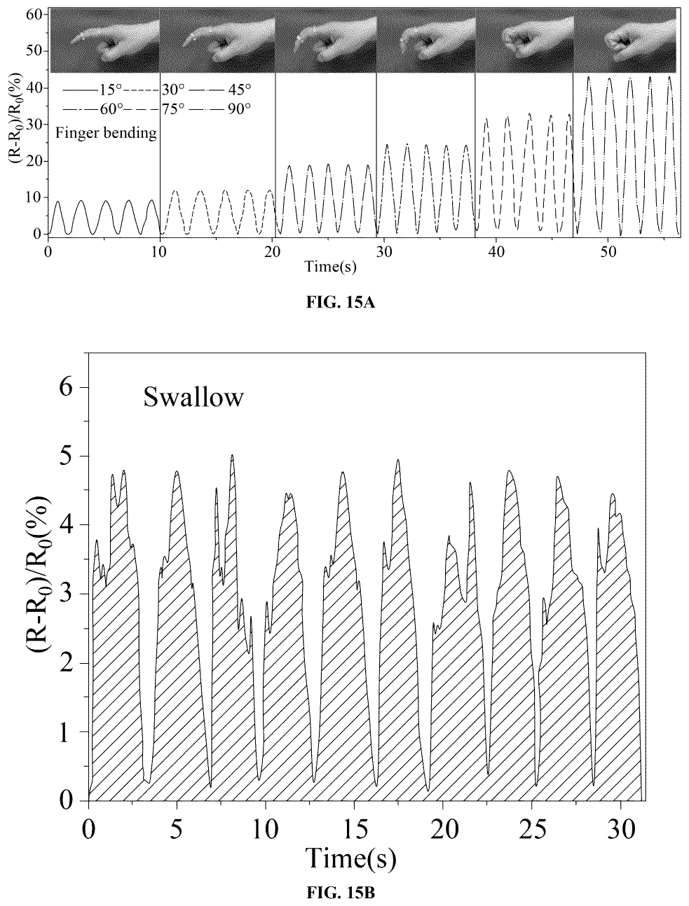

The real-time sensing performance of the ionic conductive hydrogel-based sensor prepared in Example 3 on the human body was tested by a four-probe, a digital electric bridge, and a cyclic reciprocating slide rail. The test results are shown in FIG. 15A and FIG. 15B, which are the monitoring signals of the ionic conductive hydrogel-based sensor in Example 3 for the human finger bending (FIG. 15A) and vocalization behaviors (FIG. 15B). It can be seen from FIG. 15A to FIG. 15B that the hydrogel-based sensor can accurately detect finger bending and throat vocalization behaviors of the human, and convert them into repeatable electrical signals.

What is claimed is:

1. A method for preparing a poly(ionic liquid) (PIL)-functionalized cellulose conductive hydrogel, comprising:
step 1, preparing an ionic liquid monomer A and an ionic liquid monomer P;
step 2, preparing a PIL: P(Aₓ-co-P_{y}) from the ionic liquid monomer A and the ionic liquid monomer P obtained in step 1; and
step 3, preparing the PIL-functionalized cellulose conductive hydrogel from the PIL obtained in step 2.

2. The method for preparing the PIL-functionalized cellulose conductive hydrogel according to claim 1, wherein in step 1, the ionic liquid monomer A is prepared by a process comprising:
adding 1.002 g to 9.015 g of dimethylaminoethyl acrylate (DMAEA) and 1.168 g to 10.515 g of 1-bromohexane (BH) to 5 mL to 45 mL of an acetonitrile solution, and conducting first continuous stirring and a first ultrasonic treatment until a first resulting mixture is dissolved to obtain a solution B;
subjecting the solution B to a first reaction at a temperature of 50° C. to 75° C. for 12 h to 24 h under nitrogen protection to obtain a mixture C;
adding 45 mL to 135 mL of ethyl ether in three batches to the mixture C until a white precipitate is produced; and
vacuum-drying the white precipitate to obtain the ionic liquid monomer A; and
the ionic liquid monomer P is prepared by a process comprising:
dissolving 90.4 μL to 542.4 μL of 1-vinylimidazole (VI) and 0.2149 g to 1.0742 g of 4-(bromomethyl)phenylboronic acid (BPBA) in 7.5 mL to 37.5 mL of ethyl acetate, and conducting second continuous stirring and a second ultrasonic treatment until a second resulting mixture is dissolved to obtain a solution D;
subjecting the solution D to a second reaction at a temperature of 50° C. to 75° C. for 12 h to 24 h under nitrogen protection to obtain a mixture E;
adding 22.5 mL to 112.5 mL of ethyl acetate in three batches to the mixture E, and conducting rotary evaporation at 35° C. until the ethyl acetate is evaporated to obtain a product F; and
vacuum-drying the product F to obtain the ionic liquid monomer P.

3. The method for preparing the PIL-functionalized cellulose conductive hydrogel according to claim 2, wherein the step 2 comprises:
adding 0.925 g to 1.079 g of the ionic liquid monomer A, 0.102 g to 0.463 g of the ionic liquid monomer P, and 0.052 g to 0.077 g of azobisisobutyronitrile (AIBN) to a round-bottomed flask, then adding 3.53 mL to 3.81 mL of N,N-dimethylformamide (DMF) to the round-bottomed flask, and conducting ultrasonic mixing to obtain a solution F;
subjecting the solution F to oxygen removal with nitrogen for 10 min to 30 min and then to a third reaction in an oil bath at a temperature of 50° C. to 70° C. for 2 h to 12 h to obtain a mixture G;
adding 1 mL to 15 mL of acetone in three batches to the mixture G to obtain a yellowish precipitate H; and
purifying the yellowish precipitate H by dialysis with water to obtain a purified product, and drying the purified product by a lyophilizer to obtain the P(Aₓ-co-P_{y}).

4. The method for preparing the PIL-functionalized cellulose conductive hydrogel according to claim 3, wherein the step 3 comprises:
step 3.1, preparing a cellulose solution; and
step 3.2, preparing the PIL-functionalized cellulose conductive hydrogel from the PIL obtained in step 2 and the cellulose solution obtained in step 3.1.

5. The method for preparing the PIL-functionalized cellulose conductive hydrogel according to claim 4, wherein the step 3.1 comprises:
adding 6 g to 10 g of NaOH and 9 g to 13 g of urea to 77 mL to 85 mL of deionized water, and conducting stirring and mixing to obtain a mixed solution I;
pre-cooling the mixed solution I to obtain a solution I; and
adding 2 g to 5 g of cellulose to the solution I, and stirring until the cellulose is dissolved to obtain a solution J.

6. The method for preparing the PIL-functionalized cellulose conductive hydrogel according to claim 5, wherein the step 3.2 comprises:
adding 0 g to 0.465 g of the P(Aₓ-co-P_{y}) and 0.1 mL to 1 mL of epichlorohydrin to the solution J obtained in step 3.1, and conducting stirring to obtain a solution K; and
pouring the solution K into a polytetrafluoroethylene (PTFE) mold, and subjecting the solution K to standing for 24 h, and then to solvent replacement with deionized water for 12 h to obtain the PIL-functionalized cellulose conductive hydrogel, denoted as Cell-P(Aₓ-co-P_{y}).
